(19) United States
(12) Patent Application Publication
(10) Pub. No.: US 2026/0106030 A1
(43) Pub. Date: Apr. 16, 2026

(54) Synergistic Markers for Anti-Propensity Prediction of Clinical Decision

(71) Applicant: THE HONG KONG POLYTECHNIC UNIVERSITY, Hong Kong (CN)

(72) Inventor: Wing Chi CHAN, Hong Kong (CN)

(21) Appl. No.: 19/419,235

(22) Filed: Dec. 15, 2025

Related U.S. Application Data
(63) Continuation-in-part of application No. 17/936,892, filed on Sep. 30, 2022, now abandoned.
(60) Provisional application No. 63/262,258, filed on Oct. 8, 2021.

(51) Int. Cl. G16H 50/20; G06N 20/10

(52) U.S. Cl. CPC G16H 50/20; G06N 20/10

(57) ABSTRACT

An algorithm for generating synergistic markers based on the deviation between the treatment and control groups in the association among existing markers or features is provided. Using the synergistic markers for predicting the treatment option solves the problem of treatment option propensity to the individual levels of covariates, such as patient demographics, clinical information and tumor characteristics. The synergistic markers are used in clinical decision support with an outcome prediction model developed for predicting a treatment option, enjoying following advantages. First, the synergistic markers predict the treatment option based on the inter-covariate association level instead of magnitudes of individual covariates. Such prediction gets rid of the propensity to certain covariates influencing the clinical decision. Second, a non-parametric method is used to generate the synergistic markers with many covariates, avoiding the curse of dimensionality and overfitting problem caused by a parametric model.

### CROSS-REFERENCE TO RELATED APPLICATIONS

This is a continuation-in-part application of U.S. non-provisional patent application Ser. No. 17/936,892 filed Sep. 30, 2022 which claims priority to, and the benefit of, U.S. provisional patent application Ser. No. 63/262,258 filed Oct. 8, 2021, the disclosures of which are hereby incorporated by reference in their entirety.

### LIST OF ABBREVIATIONS

AUROC area under the receiver-operating characteristics curve
CT computed tomography
KM Kaplan-Meier
NSCLC non-small-cell lung cancer
PET positron emission tomography
POB postoperative observation
RBF radial basis function
ROC receiver operating characteristics
SVC support vector classifier
SVM support vector machine
TCIA The Cancer Imaging Archive

### TECHNICAL FIELD

The present application generally relates to data-driven clinical decision support for assisting medical-treatment decision making. Particularly, the present application relates to method and system for providing clinical decision support via using synergistic markers for predicting a treatment effect of a treatment option.

### BACKGROUND

Data-driven clinical decision support of a cancer treatment option, such as adjuvant therapy, usually relies on the commonly used statistical analyses, including KM estimators, Cox regression model and logistic regression model, all of which examine the causal effect of the treatment on the clinical outcome or benefit.

In survival analysis, KM curves for two or more treatment levels are plotted and compared by the log rank test. Two treatment levels may be represented by adjuvant therapy and POB (i.e. no adjuvant therapy). The outcome may be survival or disease relapse time. The significant difference in the clinical outcome between the treatment levels can be examined by the survival analysis. For example, it was found by M. C. SALAZAR et al. (“Association of Delayed Adjuvant Chemotherapy with Survival after Lung Cancer Surgery,” JAMA Oncology, 2017 May. 1; 3(5): 610-619) that NSCLC patients who received adjuvant chemotherapy later had a significantly better survival when compared with patients treated with surgery alone. However, such analysis cannot quantify the change in survival or relapse time of a patient due to the treatment, and therefore cannot indicate the individual's benefit.

To predict the personalized treatment outcome in terms of duration or dichotomy, such as survival or recurrence, Cox regression or binominal multiple regression is modeled and implemented based on a panel of selected covariates. The candidate covariates include but are not limited to the treatment option, patient demographics, clinical information and tumor characteristics, and are sorted according to their effects on the outcome. The covariates enter or leave the model in order of their effects and the selection procedure is terminated until the designated cost function reaches a threshold value. The selected covariates, except the treatment option, are usually regarded as prognostic markers or factors (R. J. LITTLE and D. B. RUBIN, “Causal effects in clinical and epidemiological studies via potential outcomes: concepts and analytical approaches,” Annu. Rev. Public Health. 2000; 21:121-45). Instead of assuming the proportional effect of covariates on the outcomes, some recent studies explored and evaluated the application of corresponding machine learning and deep learning models for the same goals, e.g., S. A. SAPUTRO et al., “Prognostic models of diabetic microvascular complications: a systematic review and meta-analysis,” BMC Medical Research Methodology 2018. 18:24; Sci Rep 2021. 11, 1571.

The above-mentioned models, incorporated with treatment option as a covariate, could be easily trained and the inference is straightforward, in condition that the treatment assignment is randomized and independent of the other covariates in the training dataset. In practice, particularly for observational studies, the treatment is not randomized but assigned by the clinical deliberation with reference to the other covariates. Such dependence is realized from the observation that the covariate distributions could depart substantially between the treatment group and the control group. As illustrated in FIG. 1, the model thus obtained would be biased to the other covariates rather than elucidating the genuine effect of treatment on outcome.

To cope with the bias, researchers developed methods for estimating the propensity score for each subject using discriminant analysis or logistic regression of treatment option on covariates. The propensity score is aimed to obtain a valid causal inference by implementing matched-pairs study design, weighting the cases in training the model or acting as an additional covariate in the model (R. J. LITTLE and D. B. RUBIN as disclosed above; A. A. MOKDAD et al. “Adjuvant Chemotherapy vs Postoperative Observation Following Preoperative Chemoradiotherapy and Resection in Gastroesophageal Cancer: A Propensity Score-Matched Analysis,” JAMA Oncology 2018 Jan. 4(1): 31-38). However, the estimation of propensity score is susceptible to generalization errors of parametric model in small or imbalanced samples and ignores the interactions between covariates, which are also considered in treatment decision.

Therefore, it is crucial to develop an algorithmic method for synergizing a set of covariates, which could potentially affect the treatment decision, to generate markers that differentiate the within-group covariates' associations between treatment and control groups, in order to get rid of the propensity to individual covariates. There is a need to derive synergistic markers to replace the treatment option and act as additional covariates representing the genuine treatment effect in the outcome prediction model. The derived synergistic markers are usable for providing clinical decision support for assisting medical-treatment decision making.

### SUMMARY

Mathematical equations referenced in this Summary can be found in Detailed

### DESCRIPTION

A first aspect of the present invention is to provide a computer-implemented method for providing clinical decision support for assisting medical-treatment decision making.

The method comprises developing an outcome prediction model for predicting a treatment effect of a treatment option as an outcome of the model.

In developing the outcome prediction model, covariate data for training and testing the model are obtained. The covariate data is arranged as a two-dimensional array of data indexed by a plurality of covariates in a first dimension and a plurality of subjects in a second dimension. The plurality of subjects is divided into a treatment group whose subjects have been treated with the treatment option, and a non-treatment group whose subjects have not.

A distribution of covariate data of an individual covariate across the plurality of subjects is symmetrized and concentrated to a standard normal distribution such that the covariate data of the individual covariate across the plurality of subject are normalized to yield normalized covariate data of the individual covariate across the plurality of subjects. Respective normalized covariate data indexed by subjects in the treatment group collectively form a treatment-group dataset. Similarly, respective normalized covariate data indexed by subjects in the non-treatment group collectively form a non-treatment-group dataset.

The association level between every two covariates is calculated for the treatment group and the non-treatment group and their difference between two groups is also taken. The overall association level is defined as the sum of the association levels over all pairs of distinct covariates for a group. The treatment-group and non-treatment-group datasets are ordered in descending order of overall association level to thereby yield a higher-association dataset and a lower-association dataset where the higher-association dataset is higher than the lower-association dataset in overall association level.

The plurality of covariates is sorted to form an ordered list of covariates in descending order of the corresponding difference in cumulative association level between the higher-association dataset and the lower-association dataset.

Based on the higher- and lower-association datasets, an optimal number of covariates for truncating the ordered list of covariates is determined. It thereby yields an optimal list of covariates such that among different choices of number of covariates, using synergistic markers computed by combining normalized covariate data obtained for respective covariates in the optimal list maximizes a performance in predicting the treatment option over the plurality of subjects. This performance is computed as an average performance over the plurality of subjects.

Preferably, the sorting of the plurality of covariates to form the ordered list of covariates comprises: generating a matrix of covariate association level differences; and computing iteratively candidate values of cumulative association level difference for prioritizing covariates to enter into the ordered list of covariates. It is also preferable that the determining of the optimal number of covariates comprises: computing the synergistic markers corresponding to the cumulative association level for a subset of covariates in the ordered list of covariates; and determining a number of covariates such that the synergistic markers generated by the determined number of covariates achieves a maximal performance in predicting the treatment option among all possible choices of number of covariates.

Preferably, C_{T}(i,j) and C_{N}(i,j) are computed by EQNS. (2) and (3), respectively, where C_{T}(i,j) is an association level between ith and jth covariates of the treatment-group dataset, and C_{N}(i,j) is an association level between ith and jth covariates of the non-treatment-group dataset.

Preferably, the synergistic markers computed by combining normalized covariate data obtained for first m′ covariates, 2≤m′≤m, in the ordered list of covariates and for a kth subject in the plurality of subjects include first and second synergistic markers computed by EQNS. (8) and (10), respectively.

Preferably, the determining of the optimal number of covariates comprises: training a SVM with inputs s₁(k) and s₂(k) generated by the first m′ covariates in the ordered list of covariates for the kth subject and an output given by an answer of whether or not the kth subject has been treated with the treatment option, where s₁(k) and s₂(k) are the first and second synergistic markers computed for the kth subject by EQNS. (8) and (10), respectively; for each m′ value increasing from 2 to m, determining an area under a ROC curve for indicating a performance of the SVM in predicting the treatment effect, where the area is denoted by A(m′); and determining M such that A(M) is highest among A(m′) values, m′=2, . . . , m, whereby the optimal number of covariates is determined to be M.

In obtaining the covariate data for training and testing a treatment outcome prediction model, the covariate data may include clinical information, markers, features, facts, treatment received, and outcome.

Thereafter, the outcome prediction model is configured to use the synergistic markers to represent the treatment option such that in predicting the treatment effect personalized to a patient, the outcome prediction model receives patient data and the synergistic markers computed according to the patient data related to the respective covariates in the optimal list, and outputs the predicted outcome. In certain embodiments, the method further comprises predicting the treatment effect personalized to the patient by using the developed outcome prediction model. The predicting of the treatment effect personalized to the patient comprises: receiving the patient data across the respective covariates in the optimal list; normalizing the patient data to yield normalized patient data for each of the respective covariates; and computing the synergistic markers according to the normalized patient data computed for all the respective covariates.

A second aspect of the present invention is to provide a system for providing clinical decision support for assisting medical-treatment decision making.

The system comprises one or more computers configured to execute a process of providing clinical decision support for assisting medical-treatment decision making according to any of the embodiments of the disclosed method.

Other aspects of the present disclosure are disclosed as illustrated by the embodiments hereinafter.

### BRIEF DESCRIPTION OF THE DRAWINGS

FIG. 1 depicts a diagram illustrating that if treatment is not randomized such that subjects are not randomly allocated to the treatment group and the control group, an outcome prediction model would be biased to some covariates rather than elucidating the genuine effect of treatment on outcome.

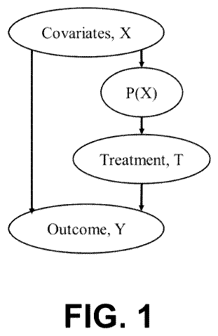

FIG. 2 depicts a flowchart showing exemplary steps used in a method of providing clinical decision support for assisting medical-treatment decision making, where the model includes a main step of developing an outcome prediction model for predicting a treatment effect of a treatment option, and an optional step of predicting a treatment effect personalized to a patient by using the developed outcome prediction model.

FIG. 3 depicts a flowchart showing steps taken for developing the outcome prediction model in accordance with an exemplary embodiment of the present invention.

FIG. 4 depicts a flowchart showing steps taken for predicting the treatment effect personalized to the patient in accordance with certain embodiments of the present invention.

FIG. 5 depicts scatter plots of covariate data for different pairs of covariates as obtained in experiments.

FIG. 6 plots two distributions of association levels of all possible covariate pairs as obtained in the experiments, where one distribution is computed for a treatment group of subjects and another one is computed for a non-treatment group.

FIG. 7 shows increasing trends of cumulative association level of higher- and lower-association datasets (as obtained from treatment-group and non-treatment-group datasets in the experiments) and their difference when the number of covariates in the ordered list increases.

FIG. 8A plots the sample means of two synergistic markers (first and second synergistic markers) and their difference against the number of covariates for the higher-association dataset.

FIG. 8B plots the sample means of the two synergistic markers and their difference against the number of covariates for the lower-association dataset.

FIG. 8C plots the sample means of the first synergistic markers for both higher- and lower-association datasets.

FIG. 8D plots the sample means of the second synergistic markers for both higher- and lower-association datasets.

FIG. 9 plots the AUROC against the number of covariates when a SVC is trained with the synergistic markers computed under different numbers of covariates in the experiments, indicating that the optimal number of covariates to be included in the optimal list of covariates for maximizing performance in predicting the treatment effect is 65.

FIG. 10A plots the ROC curve of the SVC on a training set, where the SVC was trained with the synergistic markers computed based on 65 covariates.

FIG. 10B plots the ROC curve of the SVC of FIG. 10A on a test set.

FIG. 11A plots the distributions of propensity scores for the treatment group and the non-treatment group based on the actual treatment option that was received by subjects in the experiments.

FIG. 11B plots the distributions of propensity scores for the treatment group and the non-treatment group based on the treatment predicted by the synergistic markers in the experiments.

FIG. 12 shows a user interface of the outcome prediction model for predicting a preferred treatment option for hepatocellular carcinoma (HCC), which the model was trained based on datasets containing tumor samples from HCC patients obtained at different clinical trial phrases treated with immunotherapy (atezolizumab combined with bevacizumab or atezolizumab alone) versus targeted therapy (sorafenib).

Skilled artisans will appreciate that elements in the figures are illustrated for simplicity and clarity and have not necessarily been depicted to scale.

### DETAILED DESCRIPTION

A main part of the present invention is an algorithm for generating synergistic markers based on the deviation between the treatment and control groups in the association among existing markers or features. Using the synergistic markers for predicting the effect of a treatment option solves the problem of treatment option prediction propensity to the individual levels of covariates, such as patient demographics, clinical information and tumor characteristics. The synergistic markers as disclosed herein can be advantageously used in a clinical decision support system.

A first aspect of the present invention is to provide a computer-implemented method for providing clinical decision support for assisting medical-treatment decision making.

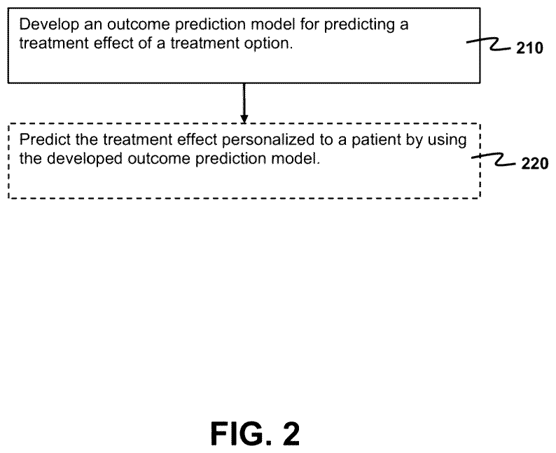

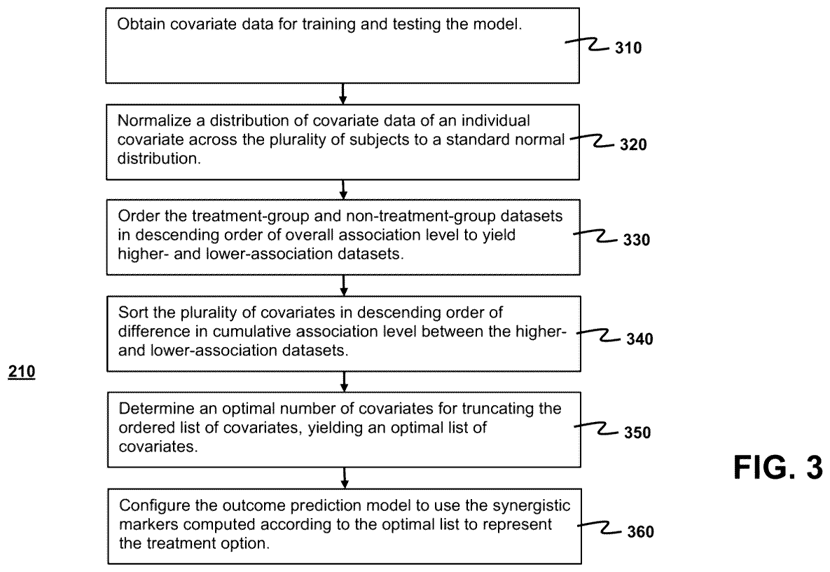

FIG. 2 depicts a flowchart showing exemplary steps of the disclosed method. In the method, an outcome prediction model for predicting a treatment effect of a treatment option as an outcome of the model is developed in step 210. The development of this model involves the derivation of the synergistic markers. The step 210 is illustrated as follows with the aid of FIG. 3, which depicts a flowchart of exemplary steps in carrying out the step 210.

For developing the outcome prediction model, covariate data for training and testing the model are first obtained in step 310. The covariate data, which include clinical information, markers, features, facts, and treatment received, are collected across a plurality of subjects to form a database. The clinical information, markers, features, and facts are model covariates. Denote xᵢ(k) as an ith covariate of a kth subject. The treatment received as collected in the covariate is used to indicate whether or not a subject in question has received treatment based on the treatment option. Note that the treatment received is intentionally not deemed to be a covariate in the development of the present invention.

Let m and n be the number of covariates and the number of subjects, respectively, as used in the database. In the database, the covariate data are arranged as a two-dimensional array of data indexed by the plurality of m covariates in a first dimension and the plurality of n subjects in a second dimension. The plurality of n subjects is divided into a treatment group whose subjects have been treated with the treatment option, and a non-treatment group whose subjects have not. Let n_{T} be the number of subjects in the treatment group, and n_{N} be the number of subjects in the non-treatment group. It follows that n=n_{T}+n_{N}.

The distributions of covariates may largely deviate from the normal distribution so that the model may be predisposed to biased prediction results if left uncorrected. Methods, such as rank-based inverse normal transformation, can be applied to symmetrize and concentrate the distribution to the standard normal distribution, N(0,1).

In step 320, a distribution of covariate data of an individual covariate across the plurality of subjects is symmetrized and concentrated to the standard normal distribution. Thus, the covariate data of the individual covariate across the plurality of subject are normalized to yield normalized covariate data of the individual covariate across the plurality of subjects. Normalization is independently applied to the covariate data of each covariate. Specifically, for each of i=1, . . . , n, the ith-covariate data (namely, the covariate data of the ith covariate) across the n subjects, i.e. xᵢ(1), xᵢ(2), . . . , xᵢ(n), are processed to symmetrize and concentrate the n covariate data's distribution to the standard normal distribution, resulting in zᵢ(1), zᵢ(2), . . . , zᵢ(n) where zᵢ(k) is denoted as a covariate data of the ith covariate, or an ith-covariate data in short. Note that the ith-covariate data across the n subjects collectively follow a near-normal distribution, ˜N(0,1). Let

$${u}_{i} = {[ {z}_{i} ( 1 ) , {z}_{i} ( 2 ) , … , {z}_{i} ( n ) ]}^{T} . \qquad ( 1 )$$

The computed values of

$${u}_{i}^{T} {u}_{i} / n and {u}_{i}^{T} {u}_{j} / n , i \neq j ,$$

tend to, respectively, l and the Pearson correlation coefficient between the ith and jth covariates when n is large enough, approaching the population size.

Denote π_{T}(K_{T}) as the position of the k_{T}th subject of the treatment group in the plurality of n subjects, where 1≤k_{T}≤n_{T}, such that the normalized ith-covariate data of this k_{T}th subject is given by zᵢ(π_{T}(k_{T})). It follows that π_{T}(k_{T}) gives an index used in the second dimension of the two-dimensional array of xᵢ(k) data corresponding to the k_{T}th subject in the treatment group. Similarly, denote π_{N}(k_{N}) as the position of the k_{N}th subject of the non-treatment group in the plurality of n subjects, where 1≤k_{N}≤n_{N}, such that the normalized ith-covariate data of this k_{N}th subject is given by zᵢ(π_{N}(k_{N})).

After the covariate data are normalized, respective normalized covariate data indexed by the n_{T} subjects in the treatment group collectively form a treatment-group dataset, and respective normalized covariate data indexed by the n_{N} subjects in the non-treatment group collectively form a non-treatment-group dataset.

Denote C_{T}(i,j) and C_{N}(i,j) as association levels between ith and jth covariates of the treatment-group dataset and of the non-treatment-group dataset, respectively, where 1≤i,j≤m. The two association levels are given by

$${C}_{T} ( i , j ) = \vert \frac{1}{{n}_{T}} {\sum }_{{k}_{T} = 1}^{{n}_{T}} {z}_{i} ( {π}_{T} ( {k}_{T} ) ) {z}_{j} ( {π}_{T} ( {k}_{T} ) ) \vert and \qquad ( 2 )$$

$${C}_{N} ( i , j ) = \vert \frac{1}{{n}_{N}} {\sum }_{{k}_{N} = 1}^{{n}_{N}} {z}_{i} ( {π}_{N} ( {k}_{N} ) ) {z}_{j} ( {π}_{N} ( {k}_{N} ) ) \vert . \qquad ( 3 )$$

Based on computed values of C_{T}(i,j) for different combinations of i and j, an overall association level of the treatment-group dataset is computed by

$${\sum }_{i \neq j , i = 1 , j = 1}^{i = m , j = m} {C}_{T} ( i , j ) .$$

Similarly, an overall association level of the non-treatment-group dataset is computed by

$${\sum }_{i \neq j , i = 1 , j = 1}^{i = m , j = m} {C}_{N} ( i , j ) .$$

Note that the overall association level for a group is given by the sum of the association levels over all pairs of distinct covariates for the group. The treatment-group and non-treatment-group datasets are further classified as a higher-association dataset H with a higher overall association level, and a lower-association dataset L with a lower overall association level, subject to the direction of the difference in overall association level given by

$$Δ = {\sum }_{i \neq j , i = 1 , j = 1}^{i = m , j = m} {C}_{T} ( i , j ) - {\sum }_{i \neq j , i = 1 , j = 1}^{i = m , j = m} {C}_{N} ( i , j ) . \qquad ( 4 )$$

If Δ≥0, the treatment-group dataset is assigned as the dataset H, and the non-treatment-group dataset as the dataset L. Otherwise, the treatment-group dataset is assigned as the dataset L, and the non-treatment-group dataset as the dataset H. The assignment of the treatment-group and non-treatment-group datasets is performed in step 330. In the step 330, the treatment-group and non-treatment-group datasets are ordered in descending order of overall association level to thereby yield the datasets H and L, where the dataset H has the overall association level higher than that of the dataset L.

In step 340, the plurality of m covariates is sorted to form an ordered list of covariates in descending order of difference in cumulative association level between the dataset H and the dataset L. The step 340 can be accomplished as follows.

Consider the difference between the datasets H and L in association level between the ith and jth covariates. This difference is formulated by the (i,j)th element of a matrix, D, computed by

$$D ( i , j ) = \{ \begin{matrix}{C}_{H} ( i , j ) - {C}_{L} ( i , j ) & for i \neq j \\ 0 & for i = j .\end{matrix} \qquad ( 5 )$$

where C_{H}(i,j) and C_{L}(i,j) are the association levels between the ith and jth covariates of the dataset H and of the dataset L, respectively. An example of D, a 5×5 matrix generated from data of five covariates A-E, is given as follows.

| | A | B | C | D | E |
|---|---|---|---|---|---|
| A | 0 | 0.875513 | 0.761413 | 0.704578 | 0.635384 |
| B | 0.875513 | 0 | 0.623233 | 0.620385 | 0.50633 |
| C | 0.761413 | 0.623233 | 0 | 0.637049 | 0.787873 |
| D | 0.704578 | 0.620385 | 0.637049 | 0 | 0.477486 |
| E | 0.635384 | 0.50633 | 0.787873 | 0.477486 | 0 |

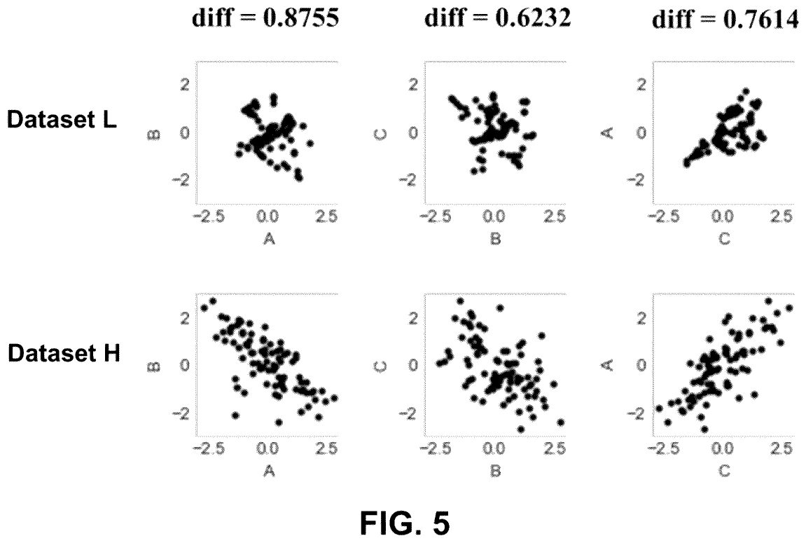

The scatter plots of (A, B), (B, C) and (C, A) of the datasets L and H are depicted in FIG. 5. From FIG. 5, it is apparent that when the association level between two covariates in the dataset L is substantially weaker than that in the dataset H, the corresponding value in D is relatively high.

Half of the off-diagonal elements of D, from either the upper or the lower triangular matrix, are extracted to form a list. The maximum of the list and the corresponding covariate pair are identified. The selected covariate list with m′ covariates is denoted by L_{m′}. For the above example of D, the maximum is 0.8755, the covariates A and B are selected and L₂ is {A, B}.

The third covariate is added to L₂ in condition that the sum of its D(i,j) values with A and B is the highest amongst the other covariates. To find the highest sum, the columns A and B of the matrix D are added element-by-element in numerical value. The result of column addition is shown below.

| | {A, B} | C | D | E |
|---|---|---|---|---|
| | 0.875513 | 0.761413 | 0.704578 | 0.635384 |
| | 0.875513 | 0.623233 | 0.620385 | 0.50633 |
| C | 1.384646 | 0 | 0.637049 | 0.787873 |
| D | 1.324963 | 0.637049 | 0 | 0.477486 |
| E | 1.141714 | 0.787873 | 0.477486 | 0 |

From the first column of the result, the covariate C yields the highest sum of D(i,j) values with A and B so that C is added to the list, giving L₃, which is {A, B, C}. To determine the fourth covariate, numerical values in columns {A, B} and C are added element-by-element to give the result below.

| | {A, B, C} | D | E |
|---|---|---|---|
| | 1.636926 | 0.704578 | 0.635384 |
| | 1.498746 | 0.620385 | 0.50633 |
| | 1.384646 | 0.637049 | 0.787873 |
| D | 1.962012 | 0 | 0.477486 |
| E | 1.929587 | 0.477486 | 0 |

From the first column again, the covariate D yields the highest sum of D(i,j) values with A, B and C so that D is added to the list, giving L₄, which is {A, B, C, D}.

For adding subsequent covariates to the list, the above steps of column addition and optimal value search are repeated. For m′ ranging from 2 to m, an ordered list of covariates can be formed in descending order of corresponding difference in cumulative association level, Δ_{m′}, given as

$${Δ}_{{m}^{′}} = {CC}_{H} ( {m}^{′} ) - {CC}_{L} ( {m}^{′} ) where \qquad ( 6 )$$

$${CC}_{H} ( {m}^{′} ) = {\sum }_{i \neq j , i = 1 , j = 1}^{i = {m}^{′} , j = {m}^{′}} {C}_{H} ( i , j ) and$$

$${CC}_{L} ( {m}^{′} ) = {\sum }_{i \neq j , i = 1 , j = 1}^{i = {m}^{′} , j = {m}^{′}} {C}_{L} ( i , j )$$

are the cumulative association levels of the dataset H and of the dataset L, respectively. Note that CC_{H}(m′) and CC_{L}(m′) each denote a respective cumulative association level calculated for first m′ covariates, 2≤m′≤m, in the ordered list of covariates.

As a summary of the above-disclosed procedure in sorting the plurality of m covariates, it is preferable that the step 340 comprises: generating a matrix of covariate association level differences; and computing iteratively candidate values of cumulative association level difference for prioritizing covariates to enter into the ordered list of covariates.

For convenience, let α(i), i∈{1, . . . , m}, be an index in the first dimension of the two-dimensional array of xᵢ(k) data corresponding to the covariate located at an ith position of the ordered list of covariates. That is, the normalized covariate data of the kth subject for the ith covariate listed in the ordered list is given by z_{α(i)}(k).

After the ordered list of m covariates is obtained, an optimal number of covariates for truncating the ordered list of m covariates is determined in step 350 to thereby yield an optimal list of covariates. In particular, the optimal number of covariates is determined such that among different choices of number of covariates, using synergistic markers computed by combining normalized covariate data obtained for respective covariates in the optimal list maximizes a performance in predicting the treatment option, where the performance is computed as an average performance over the plurality of subjects.

Before a derivation the optimal number of covariates is given, the synergistic markers are first derived.

For m′ ranging from 2 to m, the cumulative association level of the dataset H or L must fall within an interval whose lower and upper bounds are given by the sample means of two synergistic markers, s₁ and s₂. For m′ covariates, twice of the cumulative association level is elaborated to give the lower bound by an inequality to be shown. Since the datasets H and L are respective copies of either the treatment-group and non-treatment-group datasets, the treatment-group dataset is used as a representative case for illustration. The inequality related to the cumulative association level of the treatment-group dataset is given by

$$2 {\sum }_{i \neq j , i = 1 , j = 1}^{i = {m}^{′} , j = {m}^{′}} {C}_{T} ( i , j ) = 2 {\sum }_{i \neq j , i = 1 , j = 1}^{i = {m}^{′} , j = {m}^{′}} \vert \frac{1}{{n}_{T}} {\sum }_{{k}_{T} = 1}^{{n}_{T}} {z}_{α ( i )} ( {π}_{T} ( {k}_{T} ) ) {z}_{α ( j )} ( {π}_{T} ( {k}_{T} ) ) \vert \geq {\sum }_{i \neq j , i = 1 , j = 1}^{i = {m}^{′} , j = {m}^{′}} \frac{1}{{n}_{T}} {\sum }_{{k}_{T} = 1}^{{n}_{T}} 2 {z}_{α ( i )} ( {π}_{T} ( {k}_{T} ) ) {z}_{α ( j )} ( {π}_{T} ( {k}_{T} ) ) \geq \frac{1}{{n}_{T}} {\sum }_{{k}_{T} = 1}^{{n}_{T}} [ {( {\sum }_{i = 1}^{{m}^{′}} {z}_{α ( i )} ( {π}_{T} ( {k}_{T} ) ) )}^{2} - {\sum }_{i = 1}^{{m}^{′}} {( {z}_{α ( i )} ( {π}_{T} ( {k}_{T} ) ) )}^{2} ] \geq \frac{1}{{n}_{T}} {\sum }_{{k}_{T} = 1}^{{n}_{T}} {s}_{1} ( {π}_{T} ( {k}_{T} ) ) \qquad ( 7 )$$

where s₁(k) is the first synergistic marker computed for a kth subject and is defined by

$${s}_{1} ( k ) = {( {\sum }_{i = 1}^{{m}^{′}} {z}_{α ( i )} ( k ) )}^{2} - {\sum }_{i = 1}^{{m}^{′}} {( {z}_{α ( i )} ( k ) )}^{2} . \qquad ( 8 )$$

The upper bound is elaborated by the following inequality:

$$2 {\sum }_{i \neq j , i = 1 , j = 1}^{i = {m}^{′} , j = {m}^{′}} {C}_{T} ( i , j ) = 2 {\sum }_{i \neq j , i = 1 , j = 1}^{i = {m}^{′} , j = {m}^{′}} \vert \frac{1}{{n}_{T}} {\sum }_{{k}_{T} = 1}^{{n}_{T}} {z}_{α ( i )} ( {π}_{T} ( {k}_{T} ) ) {z}_{α ( j )} ( {π}_{T} ( {k}_{T} ) ) \vert \leq {\sum }_{i \neq j , i = 1 , j = 1}^{i = {m}^{′} , j = {m}^{′}} \frac{1}{{n}_{T}} {\sum }_{{k}_{T} = 1}^{{n}_{T}} 2 \vert {z}_{α ( i )} ( {π}_{T} ( {k}_{T} ) ) \vert \vert {z}_{α ( j )} ( {π}_{T} ( {k}_{T} ) ) \vert \leq \frac{1}{{n}_{T}} {\sum }_{{k}_{T} = 1}^{{n}_{T}} [ {( {\sum }_{i = 1}^{{m}^{′}} \vert {z}_{α ( i )} ( {π}_{T} ( {k}_{T} ) ) \vert )}^{2} - {\sum }_{i = 1}^{{m}^{′}} {( {z}_{α ( i )} ( {π}_{T} ( {k}_{T} ) ) )}^{2} ] \leq \frac{1}{{n}_{T}} {\sum }_{{k}_{T} = 1}^{{n}_{T}} {s}_{2} ( {π}_{T} ( {k}_{T} ) ) \qquad ( 9 )$$

where s₂(k) is the second synergistic marker computed for a kth subject and is defined by

$${s}_{2} ( k ) = {( {\sum }_{i = 1}^{{m}^{′}} \vert {z}_{α ( i )} ( k ) \vert )}^{2} - {\sum }_{i = 1}^{{m}^{′}} {( {z}_{α ( i )} ( k ) )}^{2} . \qquad ( 10 )$$

With the first and second synergistic markers s₁(k) and s₂(k), the step 350 can be accomplished by a two-step approach. First, compute the synergistic markers corresponding to the cumulative association level for a subset of covariates in the ordered list of covariates. This computation is repeated for plural subsets with different numbers of covariates. Second, determine a number of covariates such that the synergistic markers generated by the determined number of covariates achieves a maximal performance in predicting the treatment option among all possible choices of number of covariates.

The number of covariates in the ordered list to be included for generating the synergistic markers can be estimated by machine learning. If a SVM realizing a classifier is used, the classifier is trained with inputs s₁(k) and s₂(k) generated by the first m′ covariates in the ordered list of covariates for the kth subject and an output given by an answer of whether or not the kth subject has been treated with the treatment option, y(k). For each value of m′ increasing from 2 to m, the area under the ROC curve is recorded as a performance of the SVM classifier in predicting the treatment option, the area being denoted by A(m′). The optimal number of covariates, M, and thus the corresponding synergistic markers, s₁(k) and s₂(k), are identified by the highest A(m′) value, i.e. A(M).

After the optimal list of covariates is obtained, the outcome prediction model is configured in step 360 to use the synergistic markers to represent the treatment option such that in predicting the treatment effect personalized to a patient, the outcome prediction model receives patient data and the synergistic markers, and outputs the predicted outcome, where the synergistic markers are computed according to the patient data related to the respective covariates in the optimal list.

As a remark, advantages of using the synergistic markers in the disclosed method are summarized as follows. First, the synergistic markers predict the treatment option based on the inter-covariate association level instead of the magnitudes of individual covariates. Such prediction can get rid of the propensity to certain covariates influencing the clinical decision. Second, a non-parametric method is used to generate the synergistic markers with many covariates. It avoids the curse of dimensionality and overfitting problem caused by parametric model.

Some experimental results were obtained, and are used to demonstrate the effectiveness of the synergistic markers in reducing or eliminating the propensity of covariates on the actual treatment option adopted in treatment.

In the experiment, the sample data in NSCLC was retrospectively acquired from the public dataset—‘NSCLC Radiogenomic’ in TCIA. This dataset was chosen because of its availability of (1) medical imaging data (C_{T} and PET/C_{T} images), (2) adjuvant therapy option and (3) clinical data (including TNM staging, smoking status and survival outcomes recorded from follow-up monitoring). After data pre-processing, 192 cases were obtained from the dataset and 851 radiomic features representing the covariates for each case were extracted from the C_{T} images. The synergistic markers were generated from the training set of 172 cases and evaluated by the test set of 20 cases.

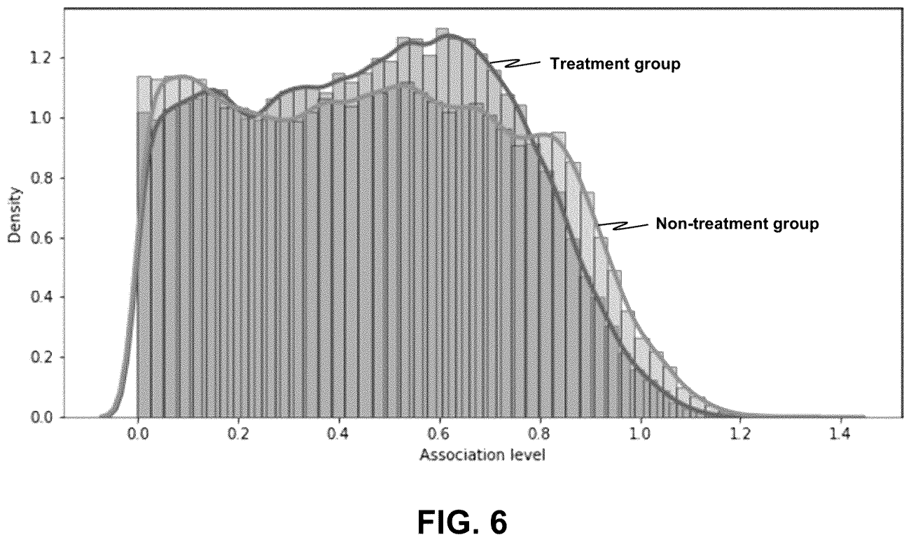

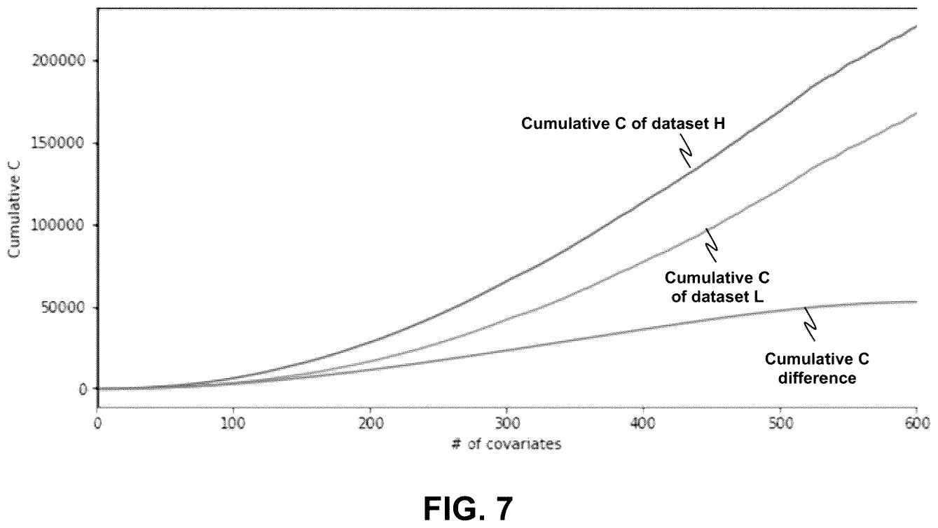

In the evaluation, the association levels of 361675 unique covariate pairs were computed for each of the treatment and non-treatment groups. Distributions of the association levels are shown and compared in FIG. 6, which plots a first distribution for the treatment group and a second distribution for the non-treatment group. The sum of association levels of the non-treatment group, 171536, is higher than that of the treatment group, 166405. The treatment group is thus defined to have dataset L and the non-treatment group to have dataset H. The covariate pair, (‘wavelet-LLH_firstorder_Median’, ‘wavelet-LLH_glcm_ClusterShade’), gave the highest difference in association level between the datasets H and L, namely, C_{H}−C_{L}. The ordered list was initialized by this pair. The subsequent covariates were added to the list one-by-one according to their cumulative association levels. FIG. 7 shows the increasing trends of cumulative association level of the datasets H and L and their difference when the number of covariates in the ordered list increases.

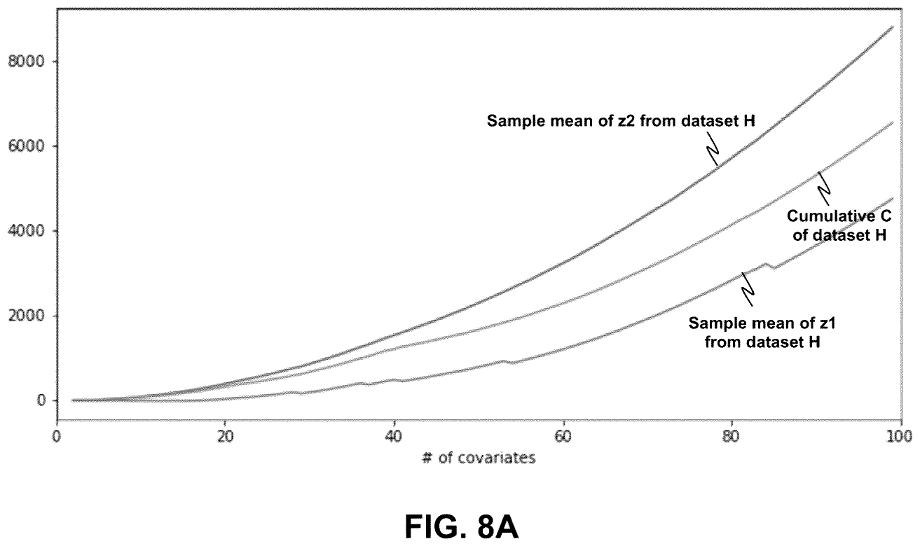

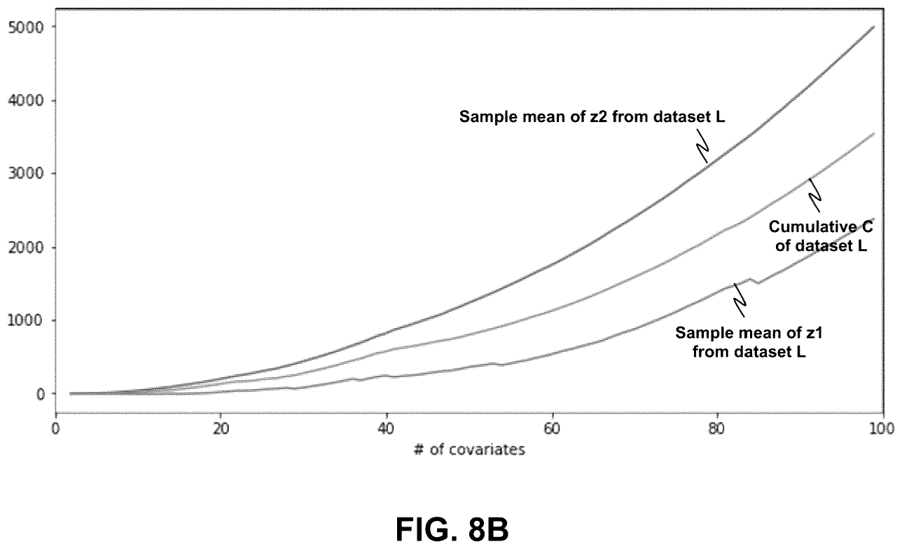

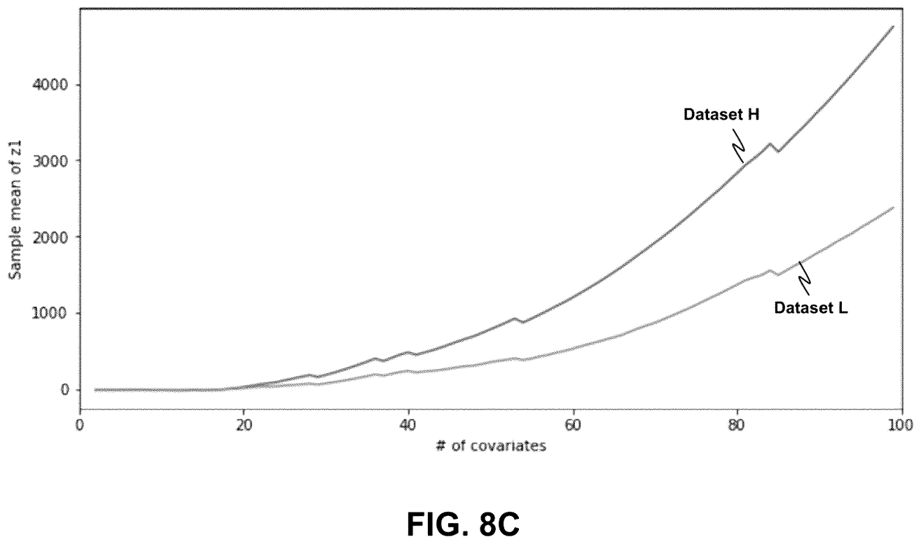

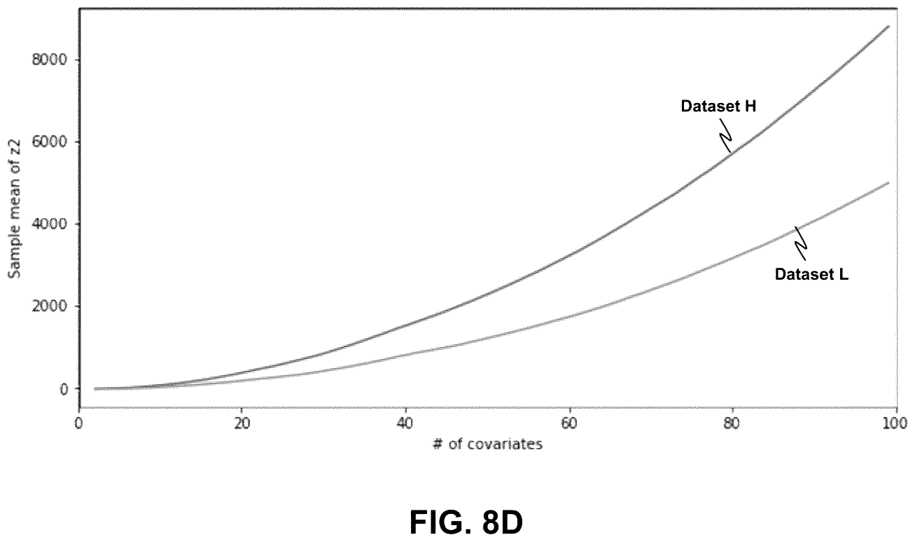

For both datasets H and L, sample means of the first and second synergistic markers z₁ and z₂ were computed. FIG. 8A plots sample means of z₁ and z₂ together with z₂−z₁ against the number of covariates for the dataset H. Similarly, FIG. 8B plots corresponding values of z₁, z₂ and z₂−z₁ against the number of covariates for the dataset L. It is apparent that the sample means of z₁ and z₂ serve as lower and upper bounds, respectively, of the cumulative association level of each of datasets H and L for any number of covariates in the ordered list. FIG. 8C plots the sample means of z₁ in datasets H and L. It is shown that the sample mean of z₁ in dataset H is higher than the corresponding sample means in dataset L and that the difference increases with the number of covariates in the ordered list. Similarly, FIG. 8D plots the sample means of z₂ in datasets H and L. The same observation is obtained.

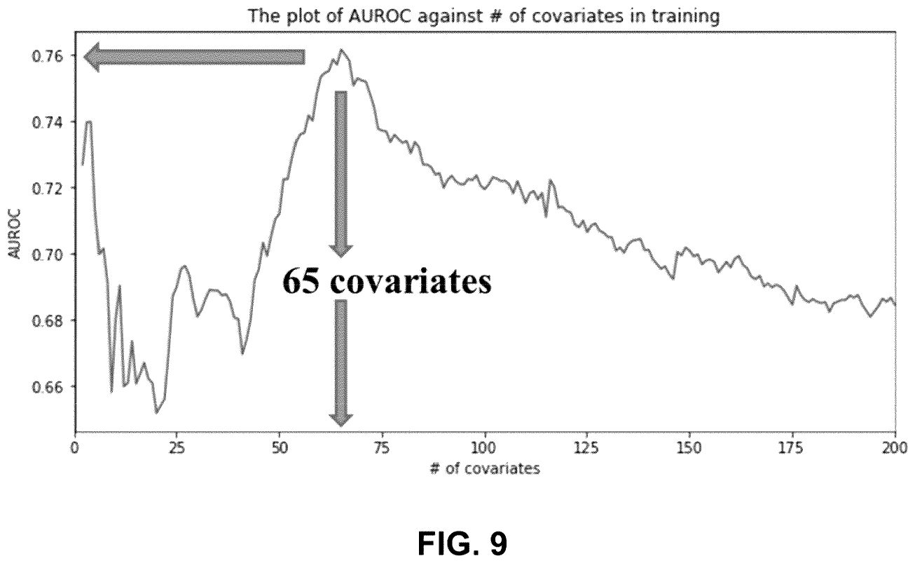

A SVC was trained with the synergistic markers, z₁ and z₂, as input and the treatment received as target output. A RBF was used as a kernel. For each covariate number, an AUROC was computed to evaluate the performance of SVC on training data. In FIG. 9, the AUROC is plotted against the number of covariates, which were used for generating the synergistic markers. It was shown that the AUROC attained the maximum, 0.76, when 65 covariates in the ordered list was used to generate the synergistic markers.

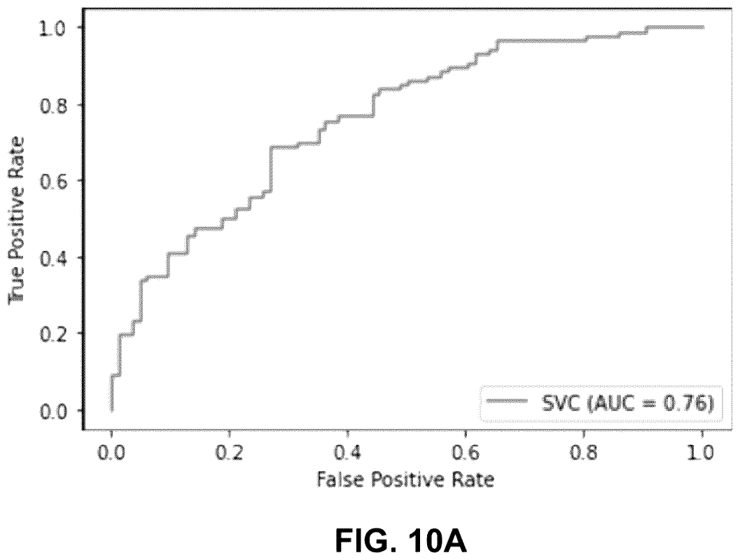

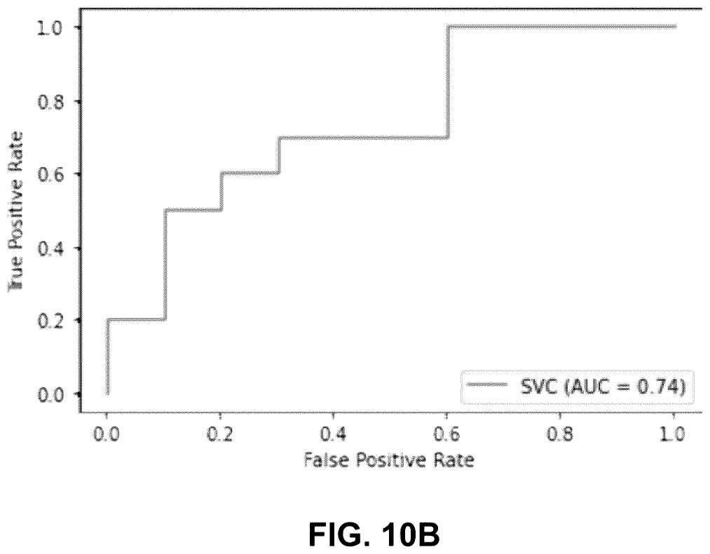

Using training and test sets, the ROC curves of the trained SVC with synergistic markers based on 65 covariates were plotted on FIGS. 10A and 10B, respectively. The test performance attained 0.74, which was close to the training performance.

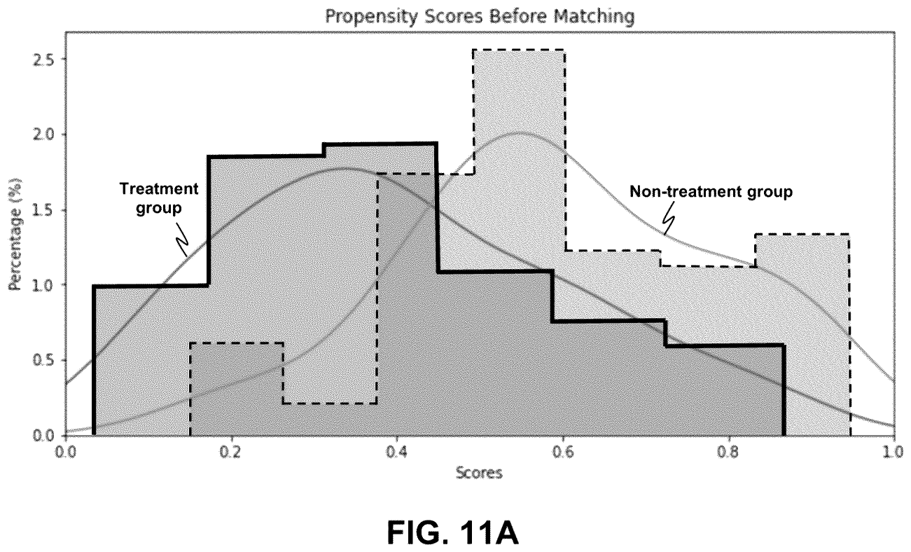

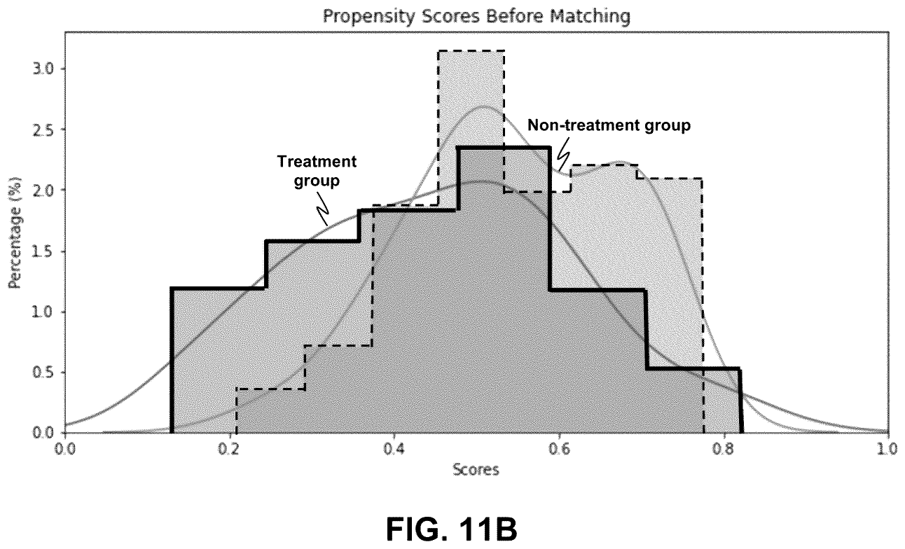

The python module, “pymatch” (https://github.com/benmiroglio/pymatch), was used to assess the propensity of covariates on the actual treatment option that was received in treatment and compare with that on the SVC prediction. The propensity scores were computed based on the first 8 covariates in the ordered list to avoid overfitting of regression model. The distributions of propensity scores were compared between treatment and non-treatment groups based on the actual treatment option received and the predicted treatment in FIGS. 11A and 11B, respectively. Significant difference in median propensity score between the treatment and non-treatment groups was found on the actual treatment option (p=2.27×10⁻⁶), but not on that predicted by the synergistic markers (p=0.08).

The experimental results demonstrate that the treatment option predicted by the synergistic markers can reduce or eliminate the propensity of covariates on the actual treatment option.

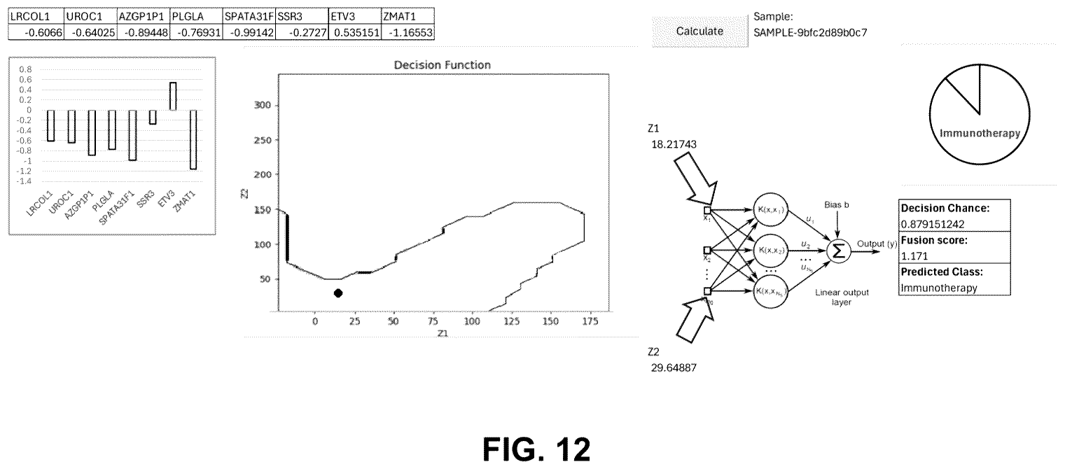

The present inventors also use the same method as in the above experiment to train a model to predict a preferred treatment option for hepatocellular carcinoma (HCC). In an exemplary embodiment, the datasets comprising genomic data of tumor samples and clinical data from patients receiving immunotherapy or targeted therapy at different clinical trial stages are used to train the model. For instance, sample data was obtained from public dataset provided by European Genome-Phenome Archive (EGA) (https://ega-archive.org/studies/EGAS00001005503). The tumor samples were collected from 358 patients enrolled in the GO30140 phase 1b or IMbrave150 phase 3 trials who were treated with atezolizumab combined with bevacizumab (immunotherapy), atezolizumab alone (immunotherapy), or sorafenib (targeted therapy). FIG. 12 shows a user interface of the model prototype prepared based on the above datasets and the method as described herein. The model can generate the probability of a treatment option, i.e., immunotherapy in this case, which is suitable for a HCC patients subject to the expression levels of 8 selected genes in the tumor sample. The corresponding synergistic markers are also calculated and illustrated as a point over the decision function on a 2D plot. The prototype can reveal the change in the synergistic markers, the point over the decision function, and the decision probability, subject to the modulation of input, i.e., the expression levels of the selected genes. This interactive feature helps identify the potential therapeutic targets that can improve the applicability of a treatment option.

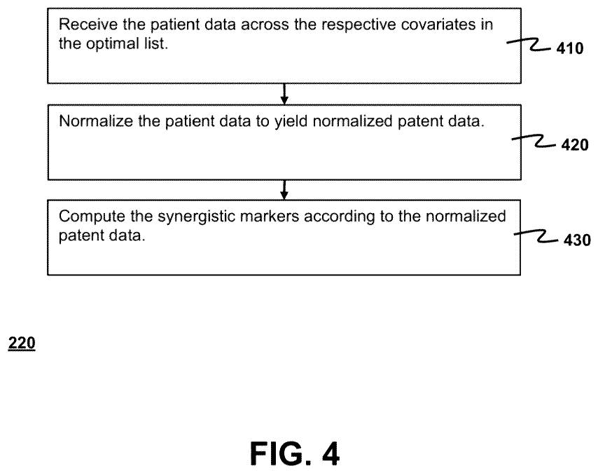

Refer to FIG. 2. Preferably and advantageously, the disclosed method further comprises the step 220 of predicting the treatment effect personalized to an individual patient by using the outcome prediction model developed in the step 210. The step 220 is illustrated as follows with the aid of FIG. 4, which depicts a flowchart of exemplary steps in carrying out the step 220.

In step 410, patient data of the individual patient across the respective covariates in the optimal list are received.

In step 420, the patient data are normalized to yield normalized patient data for each of the respective covariates. Normalization of the patient data of an individual covariate may be carried out with a mapping between a first set of xᵢ(1), xᵢ(2), . . . , xᵢ(n) values and a second set of zᵢ(1), zᵢ(2), . . . , zᵢ(n) values obtained in the step 210 where the value of i corresponds to the aforesaid individual covariate. Determining the mapping is a curve fitting problem. Those skilled in the art will appreciate that the mapping can be determined by using, e.g., interpolation formulas.

In step 430, the synergistic markers are computed according to the normalized patient data computed for all the respective covariates. The synergistic markers are used as a prediction of the treatment option in case the individual patient receives treatment based on the treatment option. As disclosed above, the synergistic markers computed for the individual patient include first and second synergistic markers. Adapted from EQNS. (8) and (10), the first and second synergistic markers are given by

$${s}_{1} ( k ) = {( {\sum }_{i = 1}^{M} {z}_{i}^{( p )} )}^{2} - {\sum }_{i = 1}^{M} {( {z}_{i}^{( p )} )}^{2} and \qquad ( 11 )$$

$${s}_{2} ( k ) = {( {\sum }_{i = 1}^{M} \vert {z}_{i}^{( p )} \vert )}^{2} - {\sum }_{i = 1}^{M} {( {z}_{i}^{( p )} )}^{2} \qquad ( 12 )$$

$${z}_{i}^{( p )}$$

is the where: s₁ is the first synergistic marker; s₂ is the second synergistic marker; patient data of the ith covariate in the optimal list determined in the step 350; and M, as mentioned above, is the number of covariates in the optimal list.

The disclosed method may be extended to evaluate respective treatment effects of plural treatment options designed for a patient. Plural sets of synergistic markers for the treatment options are obtained as indicators for predicting the respective treatment effects. A medical practitioner is thus allowed to select a preferred treatment option among the treatment options according to the obtained sets of synergistic markers. In certain embodiments, the preferred treatment option is for lung cancer or liver cancer, or associated disorders and conditions thereof. The lung cancer may include, but not limited to, non-small cell lung cancer (NSCLC); the liver cancer may include, but not limited to, hepatocellular carcinoma (HCC). Being specific for each of the indicated cancers, covariate data from a corresponding database is preferably obtained for developing an outcome prediction model. For instance, the covariate data from a radiogenomic dataset of non-small cell lung cancer is obtained for training and testing the corresponding model specific for predicting a personalized treatment effect of an individual treatment option for NSCLC patients. In certain embodiments, the radiogenomic dataset may be obtained from an open source or from in-house database/local network. For instance, the radiogenomic dataset may be obtained from ‘NSCLC Radiogenomic’ in The Cancer Imaging Archive (TCIA). The radiogenomic dataset may include, but not limited to, medical images comprising Computed Tomography (CT), Positron Emission Tomography (PET)/CT images, semantic annotations of tumors observed on the medical images using a controlled vocabulary, segmentation maps of tumors in the CT scans, adjuvant therapy option, and clinical data comprising TNM staging, smoking status and survival outcomes recorded from follow-up monitoring of patients with non-small cell lung cancer, etc. In certain embodiments, dataset specific for the other lung cancer could be obtained as the covariate data for training and testing the corresponding model for predicting a personalized treatment effect of an individual treatment option for patients with that particular lung cancer.

In certain embodiments, the NSCLC includes resected NSCLC or unresectable NSCLC. In certain embodiments, the adjuvant therapy option includes platinum-doublet chemotherapy, targeted therapy such as adjuvant epidermal growth factor receptor (EGFR) tyrosine kinase inhibitors, or immunotherapy for suppressing expression of certain EGFR mutations. In certain embodiments, the NSCLC patients may have driver mutation including EGFR mutations such as exon insertion or skipping mutations. In other words, NSCLC patients with driver mutation such as EGFR mutations are likely to be treated with corresponding targeted therapy such as tyrosine kinase inhibitors (TKIs) or immunotherapy such as specific monoclonal antibody.

Likewise, the covariate data for training and testing the corresponding model specific for predicting an individual treatment option's effect on HCC patients may include, but not limited to, datasets from a single and a combined immunotherapy or targeted therapy for advanced hepatocellular carcinoma patients in the European Genome-Phenome Archive, which are a series of datasets including genomic data from tumor samples, clinical data, and treatment response evaluation of HCC patients in different clinical trial phases treated with atezolizumab combined with bevacizumab (immunotherapy), atezolizumab alone (immunotherapy), or sorafenib (targeted therapy), respectively. Treatment response is an aspect of phenome that can be determined by the genome. Thus, the covariates include the gene expression levels, which can affect and interact with the choice of treatment option to cause the treatment outcome. Prediction of an individual treatment option's personalized treatment effect on a particular liver cancer includes obtaining a corresponding covariate data from a specific database having the corresponding dataset(s) for training and testing the corresponding model for that particular liver cancer. In certain embodiments, the HCC includes resected HCC or unresectable HCC.

A second aspect of the present invention is to provide a system for providing clinical decision support for assisting medical-treatment decision making. The system comprises one or more computers configured to execute a process of providing clinical decision support according to any of the embodiments of the method as disclosed herein. An individual computer may be a general-purpose computer, a workstation, a computing server, a distributed server in a computing cloud, a notebook computer, a mobile computing device, etc.

The present disclosure may be embodied in other specific forms without departing from the spirit or essential characteristics thereof. The present embodiment is therefore to be considered in all respects as illustrative and not restrictive. The scope of the invention is indicated by the appended claims rather than by the foregoing description, and all changes that come within the meaning and range of equivalency of the claims are therefore intended to be embraced therein.

What is claimed is:

1. A computer-implemented method for selecting a preferred treatment option for lung cancer or associated disorders and conditions thereof from a plurality of treatment options designed for a patient, the method comprising:
predicting a personalized treatment effect of an individual treatment option in the plurality of treatment options with the personalized treatment effect personalized to the patient such that respective personalized treatment effects for the plurality of treatment options are obtained; and
selecting the preferred treatment option from the plurality of treatment options according to the respective personalized treatment effects;
wherein the predicting of the personalized treatment effect of the individual treatment option with the personalized treatment effect personalized to the patient comprises:
developing an outcome prediction model for predicting a treatment effect of the individual treatment option as an outcome of the model, wherein the developing of the outcome prediction model comprises:
obtaining covariate data from a radiogenomic dataset of lung cancer for training and testing the model, the covariate data being arranged as a two-dimensional array of data indexed by a plurality of covariates in a first dimension and a plurality of subjects in a second dimension, wherein the plurality of subjects is divided into a treatment group whose subjects have been treated with the individual treatment option, and a non-treatment group whose subjects have not;
symmetrizing and concentrating a distribution of covariate data of an individual covariate across the plurality of subjects to a standard normal distribution such that the covariate data of the individual covariate across the plurality of subjects are normalized to yield normalized covariate data of the individual covariate across the plurality of subjects, whereby respective normalized covariate data indexed by subjects in the treatment group collectively form a treatment-group dataset, and respective normalized covariate data indexed by subjects in the non-treatment group collectively form a non-treatment-group dataset;
ordering the treatment-group and non-treatment-group datasets in descending order of overall association level to thereby yield a higher-association dataset and a lower-association dataset wherein the higher-association dataset is higher than the lower-association dataset in overall association level;
sorting the plurality of covariates to form an ordered list of covariates in descending order of difference in cumulative association level between the higher-association dataset and the lower-association dataset;
based on the higher- and lower-association datasets, determining an optimal number of covariates for truncating the ordered list of covariates to thereby yield an optimal list of covariates such that among different choices of number of covariates, using synergistic markers computed by combining normalized covariate data obtained for respective covariates in the optimal list maximizes a performance in predicting the individual treatment option over the plurality of subjects, the performance being computed as an average performance over the plurality of subjects; and
configuring the outcome prediction model to use the synergistic markers to represent the individual treatment option in predicting the treatment effect;
receiving patient data of the patient across the respective covariates in the optimal list;
normalizing the patient data to yield normalized patient data for the respective covariates;
computing the synergistic markers according to the normalized patient data; and
using the outcome prediction model with the synergistic markers computed according to the normalized patient data to predict the personalized treatment effect;
wherein the radiogenomic dataset of lung cancer comprises medical images comprising Computed Tomography (C_{T}), Positron Emission Tomography (PET)/C_{T} images, semantic annotations of tumors observed on the medical images using a controlled vocabulary, segmentation maps of tumors in the C_{T} scans, adjuvant therapy option, and clinical data comprising TNM staging, smoking status and survival outcomes recorded from follow-up monitoring of patients with lung cancer.

2. The method of claim 1, wherein the sorting of the plurality of covariates to form the ordered list of covariates comprises:
generating a matrix of covariate association level differences; and
computing iteratively candidate values of cumulative association level difference for prioritizing covariates to enter into the ordered list of covariates.

3. The method of claim 2, wherein the determining of the optimal number of covariates comprises:
computing the synergistic markers corresponding to the cumulative association level for a subset of covariates in the ordered list of covariates; and
determining a number of covariates such that the synergistic markers generated by the determined number of covariates achieves a maximal performance in predicting the individual treatment option among all possible choices of number of covariates.

4. The method of claim 1, wherein the overall association levels of the treatment-group dataset and of the non-treatment-group dataset are computed by
$${\sum }_{i \neq j , i = 1 , j = 1}^{i = m , j = m} {C}_{T} ( i , j ) and$$
$${\sum }_{i \neq j , i = 1 , j = 1}^{i = m , j = m} {C}_{N} ( i , j ) ,$$
respectively, where:
m is a number of covariates in the plurality of covariates;
C_{T}(i,j) is an association level between ith and jth covariates of the treatment-group dataset, given by
$${C}_{T} ( i , j ) = \vert \frac{1}{{n}_{T}} {\sum }_{{k}_{T} = 1}^{{n}_{T}} {z}_{i} ( {π}_{T} ( {k}_{T} ) ) {z}_{j} ( {π}_{T} ( {k}_{T} ) ) \vert$$
in which n_{T} is a number of subjects in the treatment-group dataset, π_{T}(k_{T}) gives an index used in the second dimension of the two-dimensional array corresponding to the k_{T}th subject in the treatment group, and zₗ(k) denotes a normalized covariate data of an Ith covariate of a kth subject in the plurality of subjects; and
C_{N}(i,j) is an association level between ith and jth covariates of the non-treatment-group dataset, given by
$${C}_{N} ( i , j ) = \vert \frac{1}{{n}_{N}} {\sum }_{{k}_{N} = 1}^{{n}_{N}} {z}_{i} ( {π}_{N} ( {k}_{N} ) ) {z}_{j} ( {π}_{N} ( {k}_{N} ) ) \vert$$
in which n_{N} is a number of subjects in the non-treatment-group dataset, and π_{N}(k_{N}) gives an index used in the second dimension of the two-dimensional array corresponding to the k_{N}th subject in the non-treatment group.

5. The method of claim 4, wherein the cumulative association levels of the higher-association dataset and of the lower-association dataset are given by
$${CC}_{H} ( {m}^{′} ) = {\sum }_{i \neq j , i = 1 , j = 1}^{i = {m}^{′} , j = {m}^{′}} {C}_{H} ( i , j ) and$$
$${CC}_{L} ( {m}^{′} ) = {\sum }_{i \neq j , i = 1 , j = 1}^{i = {m}^{′} , j = {m}^{′}} {C}_{L} ( i , j )$$
respectively, where:
CC_{H}(m′) and CC_{L}(m′) each denote a respective cumulative association level calculated for first m′ covariates, 2≤m′≤m, in the ordered list of covariates; and
C_{H}(i,j) and C_{L}(i,j) are association levels between the ith and jth covariates of the higher-association dataset and of the lower-association dataset, respectively.

6. The method of claim 1, wherein the synergistic markers computed by combining normalized covariate data obtained for first m′ covariates, 2≤m′≤m, in the ordered list of covariates and for a kth subject in the plurality of subjects include first and second synergistic markers given by
$${s}_{1} ( k ) = {( {\sum }_{i = 1}^{{m}^{′}} {z}_{α ( i )} ( k ) )}^{2} - {\sum }_{i = 1}^{{m}^{′}} {( {z}_{α ( i )} ( k ) )}^{2} and$$
$${s}_{2} ( k ) = {( {\sum }_{i = 1}^{{m}^{′}} \vert {z}_{α ( i )} ( k ) \vert )}^{2} - {\sum }_{i = 1}^{{m}^{′}} {( {z}_{α ( i )} ( k ) )}^{2} ,$$
respectively, where:
m is a length of the ordered list of covariates, and is a number of covariates in the plurality of covariates; and
α(i), i∈{1, . . . , m}, is an index of the first dimension of the two-dimensional array corresponding to the covariate located at an ith position of the ordered list of covariates.

7. The method of claim 6, wherein the determining of the optimal number of covariates comprises:
training a support vector machine (SVM) with inputs s₁(k) and s₂(k) generated by the first m′ covariates in the ordered list of covariates for the kth subject and an output given by an answer of whether or not the kth subject has been treated with the individual treatment option;
for each m′ value increasing from 2 to m, determining an area under a receiver operating characteristics (ROC) curve for indicating a performance of the SVM in predicting the individual treatment option, the area being denoted by A(m′); and
determining M such that A(M) is highest among A(m′) values, m′=2, . . . , m, whereby the optimal number of covariates is determined to be M.

8. The method of claim 1, wherein in obtaining the covariate data for training and testing the model, the covariate data include clinical information, markers, features, facts, treatment received, and outcome.

9. The method of claim 1, wherein the lung cancer comprises resected or unresectable non-small cell lung cancer.

10. A system comprising one or more computers configured to execute a process of selecting a preferred treatment option for lung cancer from a plurality of treatment options designed for a patient with said lung cancer or associated disorders and conditions thereof according to the method of claim 1.

11. A computer-implemented method for selecting a preferred treatment option for liver cancer or associated disorders and conditions thereof from a plurality of treatment options designed for a patient, the method comprising:
predicting a personalized treatment effect of an individual treatment option in the plurality of treatment options with the personalized treatment effect personalized to the patient such that respective personalized treatment effects for the plurality of treatment options are obtained; and
selecting the preferred treatment option from the plurality of treatment options according to the respective personalized treatment effects;
wherein the predicting of the personalized treatment effect of the individual treatment option with the personalized treatment effect personalized to the patient comprises:
developing an outcome prediction model for predicting a treatment effect of the individual treatment option as an outcome of the model, wherein the developing of the outcome prediction model comprises:
obtaining covariate data from a dataset of liver cancer for training and testing the model, the covariate data being arranged as a two-dimensional array of data indexed by a plurality of covariates in a first dimension and a plurality of subjects in a second dimension, wherein the plurality of subjects is divided into a treatment group whose subjects have been treated with the individual treatment option, and a non-treatment group whose subjects have not;
symmetrizing and concentrating a distribution of covariate data of an individual covariate across the plurality of subjects to a standard normal distribution such that the covariate data of the individual covariate across the plurality of subjects are normalized to yield normalized covariate data of the individual covariate across the plurality of subjects, whereby respective normalized covariate data indexed by subjects in the treatment group collectively form a treatment-group dataset, and respective normalized covariate data indexed by subjects in the non-treatment group collectively form a non-treatment-group dataset;
ordering the treatment-group and non-treatment-group datasets in descending order of overall association level to thereby yield a higher-association dataset and a lower-association dataset wherein the higher-association dataset is higher than the lower-association dataset in overall association level;
sorting the plurality of covariates to form an ordered list of covariates in descending order of difference in cumulative association level between the higher-association dataset and the lower-association dataset;
based on the higher- and lower-association datasets, determining an optimal number of covariates for truncating the ordered list of covariates to thereby yield an optimal list of covariates such that among different choices of number of covariates, using synergistic markers computed by combining normalized covariate data obtained for respective covariates in the optimal list maximizes a performance in predicting the individual treatment option over the plurality of subjects, the performance being computed as an average performance over the plurality of subjects; and
configuring the outcome prediction model to use the synergistic markers to represent the individual treatment option in predicting the treatment effect;
receiving patient data of the patient across the respective covariates in the optimal list;
normalizing the patient data to yield normalized patient data for the respective covariates;
computing the synergistic markers according to the normalized patient data; and
using the outcome prediction model with the synergistic markers computed according to the normalized patient data to predict the personalized treatment effect;
wherein the dataset of liver cancer comprises genomic data from tumor samples, clinical data, and treatment response evaluation of patients with said liver cancer.

12. The method of claim 11, wherein the sorting of the plurality of covariates to form the ordered list of covariates comprises:
generating a matrix of covariate association level differences; and
computing iteratively candidate values of cumulative association level difference for prioritizing covariates to enter into the ordered list of covariates.

13. The method of claim 12, wherein the determining of the optimal number of covariates comprises:
computing the synergistic markers corresponding to the cumulative association level for a subset of covariates in the ordered list of covariates; and
determining a number of covariates such that the synergistic markers generated by the determined number of covariates achieves a maximal performance in predicting the individual treatment option among all possible choices of number of covariates.

14. The method of claim 11, wherein the overall association levels of the treatment-group dataset and of the non-treatment-group dataset are computed by
$${\sum }_{i \neq j , i = 1 , j = 1}^{i = m , j = m} {C}_{T} ( i , j ) and$$
$${\sum }_{i \neq j , i = 1 , j = 1}^{i = m , j = m} {C}_{N} ( i , j ) ,$$
respectively, where:
m is a number of covariates in the plurality of covariates;
C_{T}(i,j) is an association level between ith and jth covariates of the treatment-group dataset, given by
$${C}_{T} ( i , j ) = \vert \frac{1}{{n}_{T}} {\sum }_{{k}_{T} = 1}^{{n}_{T}} {z}_{i} ( {π}_{T} ( {k}_{T} ) ) {z}_{j} ( {π}_{T} ( {k}_{T} ) ) \vert$$
in which n_{T} is a number of subjects in the treatment-group dataset, π_{T}(k_{T}) gives an index used in the second dimension of the two-dimensional array corresponding to the k_{T}th subject in the treatment group, and zₗ(k) denotes a normalized covariate data of an Ith covariate of a kth subject in the plurality of subjects; and
C_{N}(i,j) is an association level between ith and jth covariates of the non-treatment-group dataset, given by
$${C}_{N} ( i , j ) = \vert \frac{1}{{n}_{N}} {\sum }_{{k}_{N} = 1}^{{n}_{N}} {z}_{i} ( {π}_{N} ( {k}_{N} ) ) {z}_{j} ( {π}_{N} ( {k}_{N} ) ) \vert$$
in which n_{N} is a number of subjects in the non-treatment-group dataset, and π_{N}(k_{N}) gives an index used in the second dimension of the two-dimensional array corresponding to the k_{N}th subject in the non-treatment group.

15. The method of claim 14, wherein the cumulative association levels of the higher-association dataset and of the lower-association dataset are given by
$${CC}_{H} ( {m}^{′} ) = {\sum }_{i \neq j , i = 1 , j = 1}^{i = {m}^{′} , j = {m}^{′}} {C}_{H} ( i , j ) and$$
$${CC}_{L} ( {m}^{′} ) = {\sum }_{i \neq j , i = 1 , j = 1}^{i = {m}^{′} , j = {m}^{′}} {C}_{L} ( i , j )$$
respectively, where:
CC_{H}(m′) and CC_{L}(m′) each denote a respective cumulative association level calculated for first m′ covariates, 2≤m′≤m, in the ordered list of covariates; and
C_{H}(i,j) and C_{L}(i,j) are association levels between the ith and jth covariates of the higher-association dataset and of the lower-association dataset, respectively.

16. The method of claim 11, wherein the synergistic markers computed by combining normalized covariate data obtained for first m′ covariates, 2≤m′≤m, in the ordered list of covariates and for a kth subject in the plurality of subjects include first and second synergistic markers given by
$${s}_{1} ( k ) = {( {\sum }_{i = 1}^{{m}^{′}} {z}_{α ( i )} ( k ) )}^{2} - {\sum }_{i = 1}^{{m}^{′}} {( {z}_{α ( i )} ( k ) )}^{2} and$$
$${s}_{2} ( k ) = {( {\sum }_{i = 1}^{{m}^{′}} \vert {z}_{α ( i )} ( k ) \vert )}^{2} - {\sum }_{i = 1}^{{m}^{′}} {( {z}_{α ( i )} ( k ) )}^{2} ,$$
respectively, where:
m is a length of the ordered list of covariates, and is a number of covariates in the plurality of covariates; and
α(i), i∈{1, . . . , m}, is an index of the first dimension of the two-dimensional array corresponding to the covariate located at an ith position of the ordered list of covariates.

17. The method of claim 16, wherein the determining of the optimal number of covariates comprises:
training a support vector machine (SVM) with inputs s₁(k) and s₂(k) generated by the first m′ covariates in the ordered list of covariates for the kth subject and an output given by an answer of whether or not the kth subject has been treated with the individual treatment option;
for each m′ value increasing from 2 to m, determining an area under a receiver operating characteristics (ROC) curve for indicating a performance of the SVM in predicting the individual treatment option, the area being denoted by A(m′); and
determining M such that A(M) is highest among A(m′) values, m′=2, . . . , m, whereby the optimal number of covariates is determined to be M.

18. The method of claim 11, wherein in obtaining the covariate data for training and testing the model, the covariate data include clinical information, markers, features, facts, treatment received, and outcome.

19. The method of claim 11, wherein the liver cancer comprises resected or unresectable hepatocellular carcinoma.

20. A system comprising one or more computers configured to execute a process of selecting a preferred treatment option for liver cancer from a plurality of treatment options designed for a patient with said liver cancer or associated disorders and conditions thereof according to the method of claim 11.
